(19) United States
(12) Patent Application Publication
(10) Pub. No.: US 2008/0108065 A1
(43) Pub. Date: May 8, 2008

(54) SYSTEMS AND DIAGNOSTIC METHODS FOR BREAST CANCER USING MMP-1 MARKERS

(76) Inventor: Indra Poola, Washington, DC (US)

Correspondence Address: ROTHWELL, FIGG, ERNST & MANBECK, P.C., 1425 K STREET, N.W., SUITE 800, WASHINGTON, DC, 20005, US

(21) Appl. No.: 11/741,437

(22) Filed: Apr. 27, 2007

Related U.S. Application Data
(60) Provisional application No. 60/795,761, filed on Apr. 28, 2006.

(51) Int. Cl. C12Q 1/68

(52) U.S. Cl. 435/6

(57) ABSTRACT

The present disclosure relates to methods of diagnosing cancer and pre-cancerous conditions in human breast. The disclosure further relates to methods of prognosticating the likely success of clinical treatments for breast cancer. The disclosure further relates to kits suitable for performing the present methods.

### CROSS-REFERENCE TO RELATED APPLICATION

The present application claims the priority of provisional application 60/795,761, filed Apr. 28, 2006, the entire contents of which is specifically incorporated by reference for all purposes.

### STATEMENT OF FEDERAL SPONSORSHIP

The U.S. Government has a paid-up license in this invention and the right in limited circumstances to require the patent owner license others on reasonable terms as provided for by the terms of Grant Number R33 CA 88347 awarded by NIH, NCI and Grant Number DAMD 17-02-1-0409 awarded by DOD.

### FIELD OF THE INVENTION

The present disclosure relates generally to breast cancer. The disclosure relates more specifically to the use of MMP-1 as a diagnostic and predictive marker for the presence and clinical course of breast cancer.

### BACKGROUND

The information provided below is not admitted to be prior art to the present invention, but is provided solely to assist the understanding of the reader.

To identify putative markers of IBC, we compared the global gene expression profiles of six ADHC tissues (from patients with atypical ductal hyperplasia (ADH) who had cancer concurrently, who had cancer before diagnosis of ADH or who developed cancer subsequently) against ten ADH control tissues from patients who had no cancer before ADH diagnosis and did not develop cancer in the 5 years after diagnosis. ADH samples were selected for this analysis because women with ADH have approximately 5 times higher incidence of IBC than women who did not have ADH2)3. All ADH samples were derived from biopsy tissues and ADHC samples were derived from either biopsy or mastectomy tissues (for details see Poola I, et al., Identification of MMP-1 as a Putative Breast Cancer Predictive Marker By Global Gene Expression Analysis, 11 Nature Medicine, 481, (1 May 2005) (Poola 2005) and Table 2, the entire contents of which is specifically incorporated by reference). The fresh tissues for expression analyses were obtained by harvesting the lesions immediately adjacent to the tissue used for histological diagnosis and were considered to be representative of the tissue utilized for diagnosis. We performed four analyses for each group on U133A (Affymetrix) gene chip using individual or pooled RNA samples (GEO accession number GSE2429). By comparing the global gene expression profiles of ADHC and ADH, we identified about 540 differentially expressed genes in ADHC and statistically analyzed this expression (Poola, 2005). The raw data, comparisons and differentially expressed genes are presented in Poola (2005) Table 3. Validation of the expression of selected gene mRNAs by real-time RT-PCR, hierarchical clustering and over-represented gene ontologies are shown in FIG. 2, FIG. 3 and Table 4, respectively (Poola, 2005). Among the 540 genes identified, several were previously reported to be differentially expressed in breast cancer 4 and include 10 (ANKT, CENPA, TOPK, RRM2, TOM, NEK2, CDKN3, BUBI, BIRC5 and CKS2) of the 38 genes that were previously reported to be upregulated during breast cancer progressions.

The differentially expressed genes in ADHC encode for proteins that regulate at least 11 major categories of cellular functions (Table 5). The most significantly (P<10-4) upregulated mRNAs were those encoding proteins involved in: (i) regulating cell cycle check points((ii) increasing nucleic acid levels((iii) increasing estrogen levels((iv) degrading extracellular matrix (ECM) ((v) maintaining cell polarity and architecture ((vi) increasing cellular proliferation ((vii) inhibiting apoptosis and (viii) EGFR signaling. Other elevated mRNAs include those encoding for proto-oncogenes and those expressed by B and T lymphocytes. The most significantly (P<10-4) downregulated mRNAs were those encoding tumor suppressors and growth factor binding proteins.

MMP1, which encodes an enzyme that degrades extracellular matrix, was one of the most highly upregulated mRNAs in ADHC and elevated levels of MMP-1 protein have been reported in several cancers including breast cancer 6. Animal studies have suggested that overexpression of MMP-1 protein has a role in initiating mammary tumorigenesis by degrading stroma and releasing growth factors and other mitogens for epithelial cells 8, 9. MMP-1 was shown to specifically degrade insulin-like growth factor (IGF) binding proteins 2, 3 and 5, fibroblast growth factor (FGF) binding protein and transforming growth factor (TGF)-p binding protein and release IGF, FGF and TGF-R1°. ECM degradation by MMPs including MMP-1 was also shown to perturb cell-cell and cell-ECM interactions and disassociate cells from ECM, leading to increased cell division, decreased apoptosis and tumorigenesis.

We analyzed MMP-1 protein expression in a total of 103 archival precancerous tissues by immunohistochemistry. Of the 103 tissues, 30 were ADH from patients who had no cancer before and did not develop cancer in 5-7 years after diagnosis. The other 73 precancerous tissues were derived from four categories of patients with a history of cancer: (i) 14 patients with ADHC and who later developed cancer ((ii) 17 patients with non-ADH benign lesions who later developed cancer((iii) 12 patients who first had cancer and later developed ADH and (iv) 30 patients with ADHC and cancer simultaneously. Using immunohistochemistry, we found that 26 out of 30 tissues in the control group were negative for MMP-1. In the test group, 63 of the 73 samples were positive. Details about tissues used and scoring of MMP-1 staining intensities, which were graded in comparison to an arbitrary score of 5 in both epithelial cells and stroma of invasive cancer tissues, are presented in Table 6. Statistical analysis of the above data showed a significant (P−2.7×10⁻⁹) association of MMP-1 protein expression with precancerous tissues obtained from patients with a history of cancer by both chisquared and Fisher exact tests. Representative micrographs of normal, MMP-1-positive ductal hyperplasia with fibrocystic changes, ADH, ADHC, IBC and ductal carcinoma in situ (DCIS) tissues are presented in FIG. 1a. Both stroma and epithelial cells were positive for MMP-1. More intense staining was observed in stroma in all the tissues. Among various samples tested, most intense staining was observed in ADHC from patients who simultaneously had cancer and weakest was in non-ADH benign lesions (Table 6) (Poola, 2005).

We next tested the feasibility of detecting MMPI mRNA in a total of 31 ductal lavage samples by real-time RT-PCR. Of the 8 samples diagnosed as atypia, 6 were positive and 20 of the 22 diagnosed as benign were negative for MMPI mRNA (Table 1). MMPI mRNA levels in ductal lavage samples were comparable to the levels detected in IBC and DCIS samples (FIG. 1b). These results show that MMPI mRNA can be detected in cells obtained from ductal lavage.

The data presented here suggest that MMP-1 could potentially be used as a diagnostic marker for screening ADH and non-ADH benign tissues and identifying patients with lesions that may develop into cancer. It also may prove useful for screening women with no lesions using samples of ductal cells obtained by procedures such as ductal lavage collection and random periareolar fine needle aspiration and identifying those at risk of cancer development. Finally, MMP-1 upregulation in a precancerous lesion may suggest a need for treatment similar to IBC.

Other objects and advantages will become apparent from the following disclosure.

### SUMMARY OF INVENTION

An aspect of the present invention provides a method of diagnosing breast cancer in a human patient, according to an aspect, the level of gene expression is determined on a tissue sample obtained from a patient. According to a further aspect, sample of breast tissue is obtained. According to a yet further aspect, a determination is made of the level of expression from of a least one gene which may be any or all of MMP-1, HEC, CEACAM5, BCL2A1, or any other gene shown to be upregulated in breast cancer.

According to an aspect of the present invention, a method of diagnosing breast cancer comprises detecting a differential level of expression of at least one selected gene relative to a housekeeping gene. According to a further aspect, the selected gene is one known to be upregulated in cancerous or pre-cancerous tissue. According to a preferred aspect, the selected gene may be any or all of MMP-1, HEC, CEACAM5, BCL2A1, or any other gene shown to be upregulated in breast cancer. According to an aspect, a suitable, but not limiting housekeeping gene is glyceraldehyde phosphate dehydrogenase (GAPDH).

According to an aspect of the present invention, a method of diagnosing breast cancer in a human patient comprises contacting a human gene array with a human breast tissue sample. According to an aspect, the human gene array comprises at least one of the genes for MMP-1, HEC, CEACAM5, BCL2A1, or any other gene shown to be upregulated in breast cancer.

According to an aspect of the present invention, a method of diagnosing breast cancer in a human patient comprises contacting a nucleic acid extracted from a human breast tissue sample with a PCR reaction in the presence of at least one primer or probe specific for at least one of the genes for MMP-1, HEC, CEACAM5, BCL2A1, or any other gene shown to be upregulated in breast cancer. According to an aspect, the nucleic acid is an mRNA for at least one of the genes for MMP-1, HEC, CEACAM5, BCL2A1, or any other gene shown to be upregulated in breast cancer.

According to an aspect of the present invention, a method of diagnosing breast cancer in a human patient comprises contacting a nucleic acid reverse transcribed from a human breast tissue sample with a PCR reaction in the presence of at least one primer or probe specific for at least one of the genes for MMP-1, HEC, CEACAM5, BCL2A1, or any other gene shown to be upregulated in breast cancer. According to an aspect, the nucleic acid is an mRNA for at least one of the genes for MMP-1, HEC, CEACAM5, BCL2A1, or any other gene shown to be upregulated in breast cancer.

According to an aspect the present invention provides a method of monitoring the progress of a breast cancer clinical treatment. Aspects of the method comprise providing a patient with a clinical treatment, obtaining a tissue sample from said patient, and detecting the level of expression in a said tissue sample of a least one gene selected from the group consisting of MMP-1, HEC, CEACAM5, and BCL2A1, or any other gene shown to be upregulated in breast cancer. According to an aspect, a differential level of expression of at least one selected gene is detected in relation to the level of expression of a housekeeping gene.

According to an aspect the present invention provides a kit for diagnosing breast cancer comprising at least one component selected from the group consisting of a gene array chip, a breast cancer-specific gene array chip, a breast cancer-specific PCR primer, and a breast cancer-specific PCR probe. According to an aspect, the breast cancer-specific gene array chip comprises at least on gene selected from the group consisting of MMP-1, HEC, CEACAM5, and BCL2A1, or any other gene shown to be upregulated in breast cancer.

Still other aspects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 displays MMP-1 protein levels in breast tissues by IHC, MMPI mRNA in ductal lavage by quantitative real-time RT PCR;

FIG. 2 displays Validation of gene expression by micro-arrays using quantitative real-time PCR, and;

FIG. 3 depicts hierarchical clustering of unique genes having top 35 and bottom 35 ADHC/ADH ratios and 5 interesting genes (BCL.2A1, BIRC1, TACC3, CEACAM5, and TYMS) by average linkage and centered correlation. Hierarchical clustering use 1-ρ as distance metric, where ρ is Pearson correlation coefficient is shown. Logarithmic signal values were mean centered for each gene. Each point in space represents one array. The cluster is color coded using red for up-regulation and green for down regulation and black for medium expression.

### DETAILED DESCRIPTION OF A PREFERRED EMBODIMENT

Reference is made to the figures to illustrate selected embodiments and preferred modes of carrying out the invention. It is to be understood that the invention is not hereby limited to those aspects depicted in the figures.

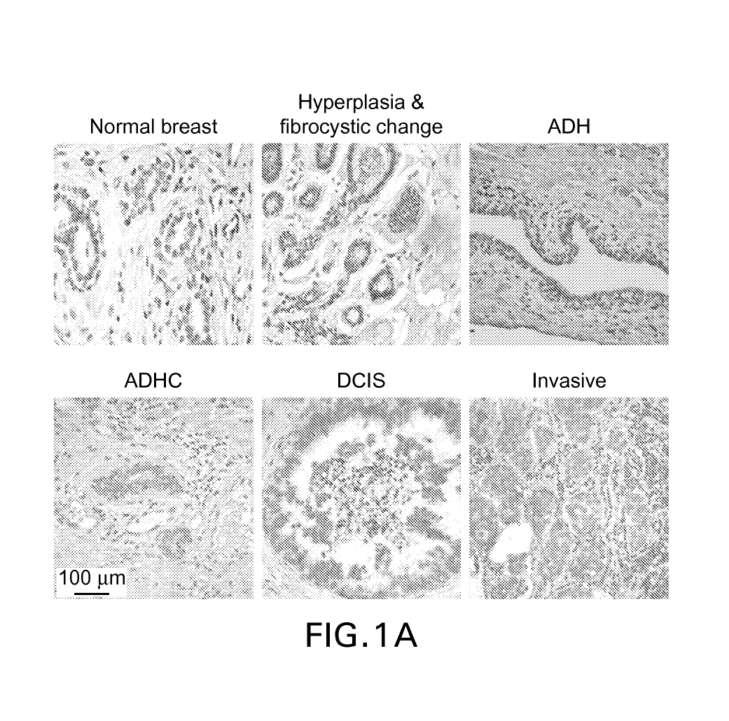

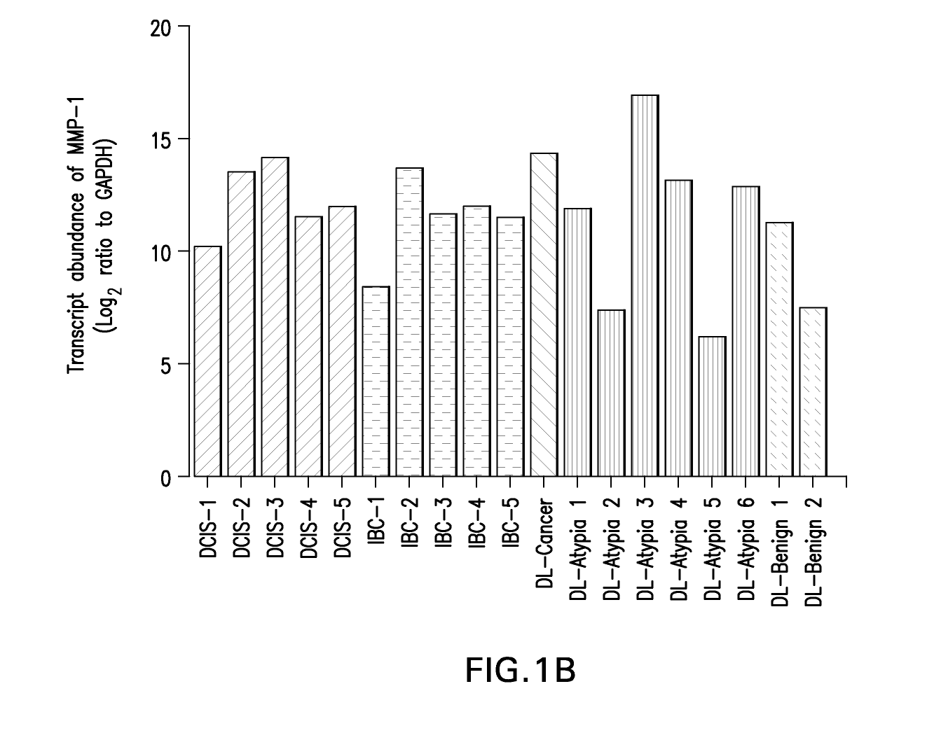

FIG. 1 MMP-1 protein levels in breast tissues by IHC, MMPI mRNA in ductal lavage by quantitative real-time RT PCR. (a) Paraffin-embedded tissue sections were immunostained by investigators blinded to the experimental conditions using standard procedures with monoclonal antibodies against MMP-1. Representative tissues stained from normal, MMP-1-positive nonADH benign tissue (hyperplasia with fibrocystic change), ADH, ADHC, DCIS and IBC are shown (magnification, ×100). MMP-1 staining could be seen both in ductal epithelial cells and stroma. (b) Feasibility of detecting MMPI mRNA was tested in a total of 31 ductal lavage samples by quantitative realtime RTPCR using Assays-on-Demand reagents from Applied Biosystems, Inc. The ductal lavage samples were obtained from donors using InDuct breast micro-catheters from Cytyc and total RNAs were isolated using Qiagen RNeasy Micro Kits. MMP1 transcript levels in nine positive ductal lavage, five representative DCIS and Stage I IBC samples are shown as histograms. Six samples diagnosed as having atypia (DL-Atypia 1-6) and two samples diagnosed as benign (DL-Benign 1-2) showed comparable transcript levels as DCIS and Stage I samples. Ductal lavage collection procedure was approved by the Howard University Institutional Review Board Committee.

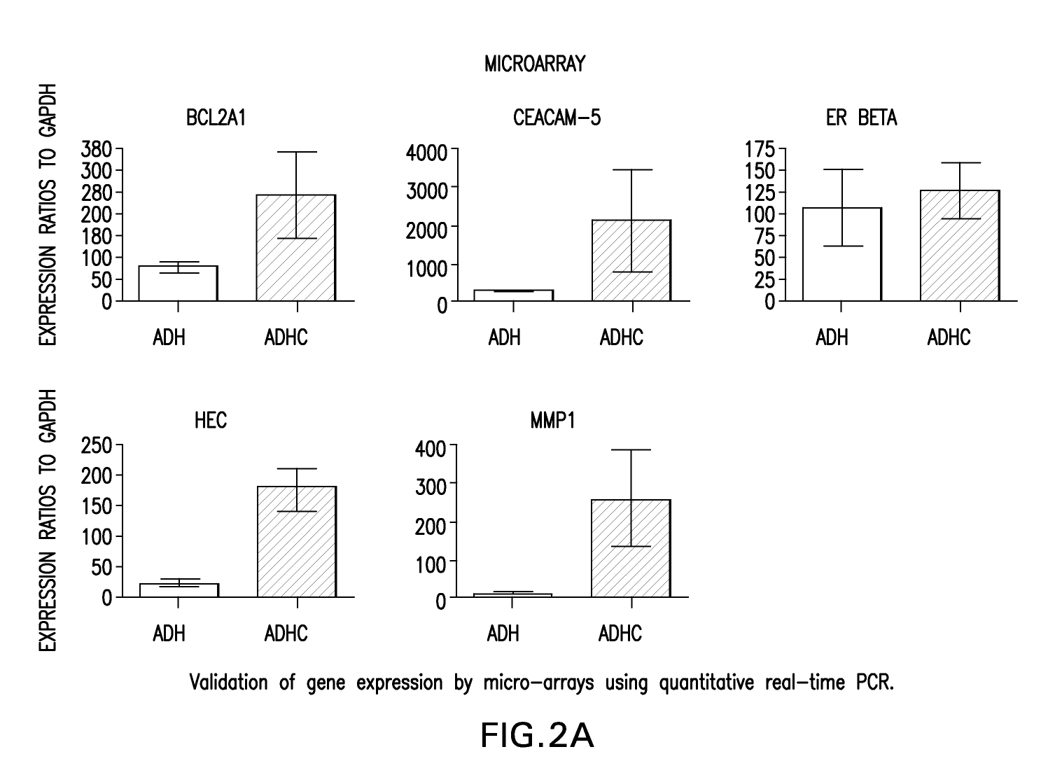

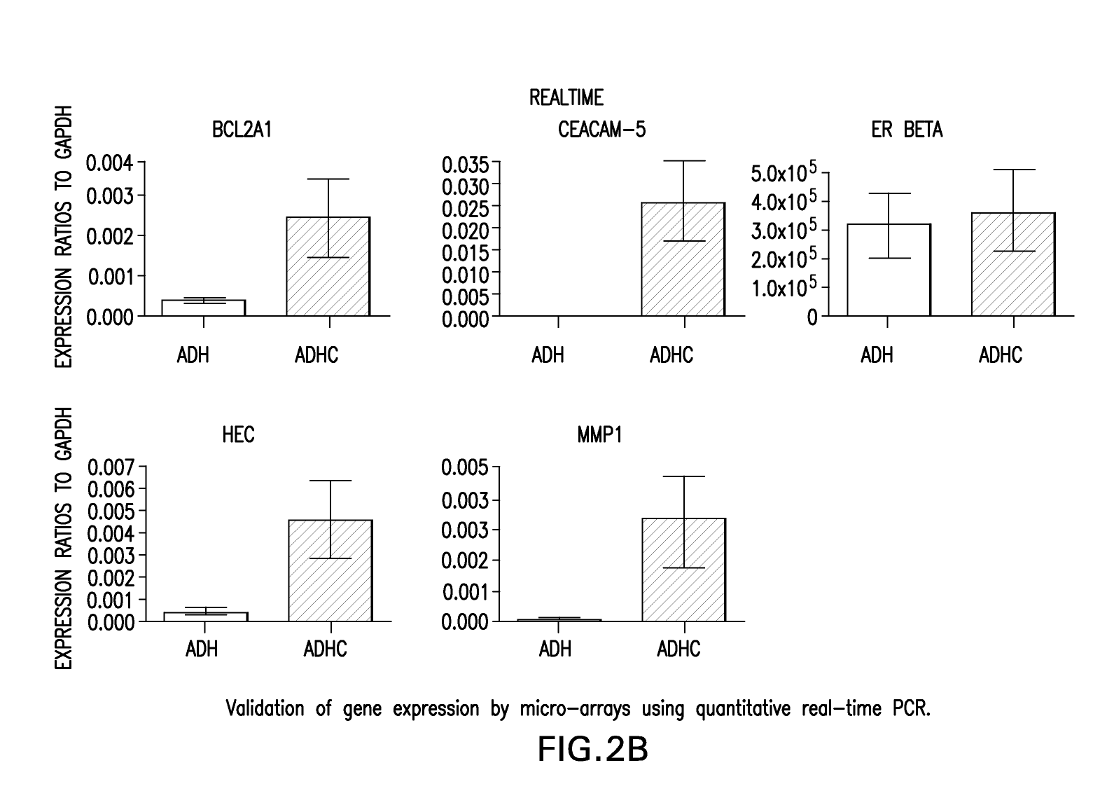

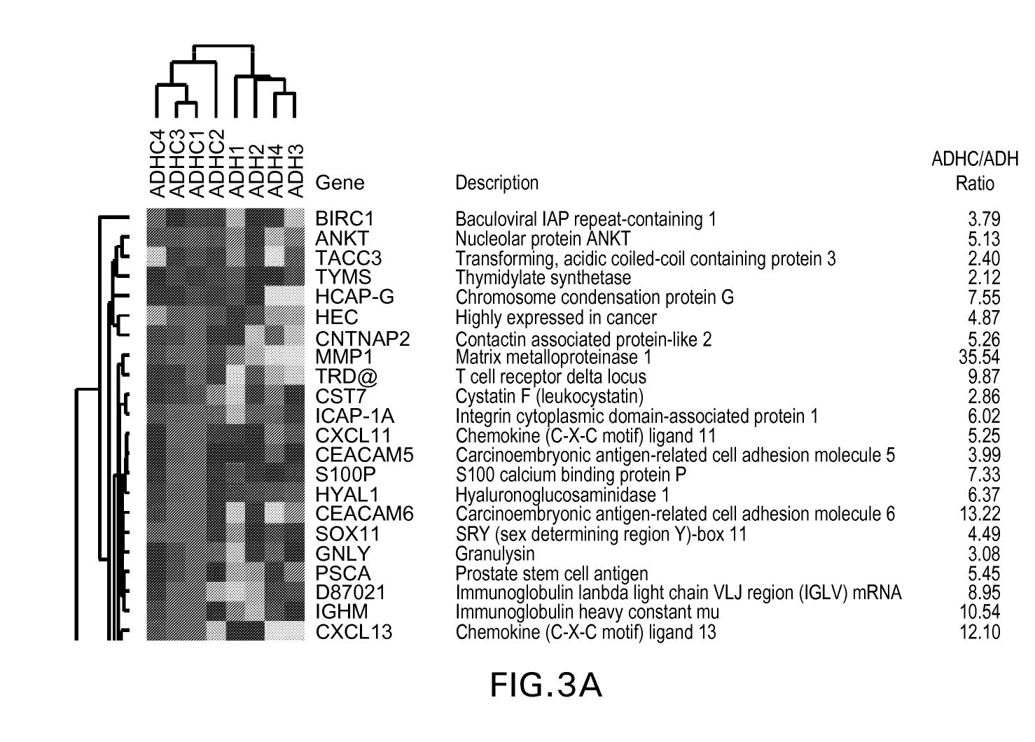

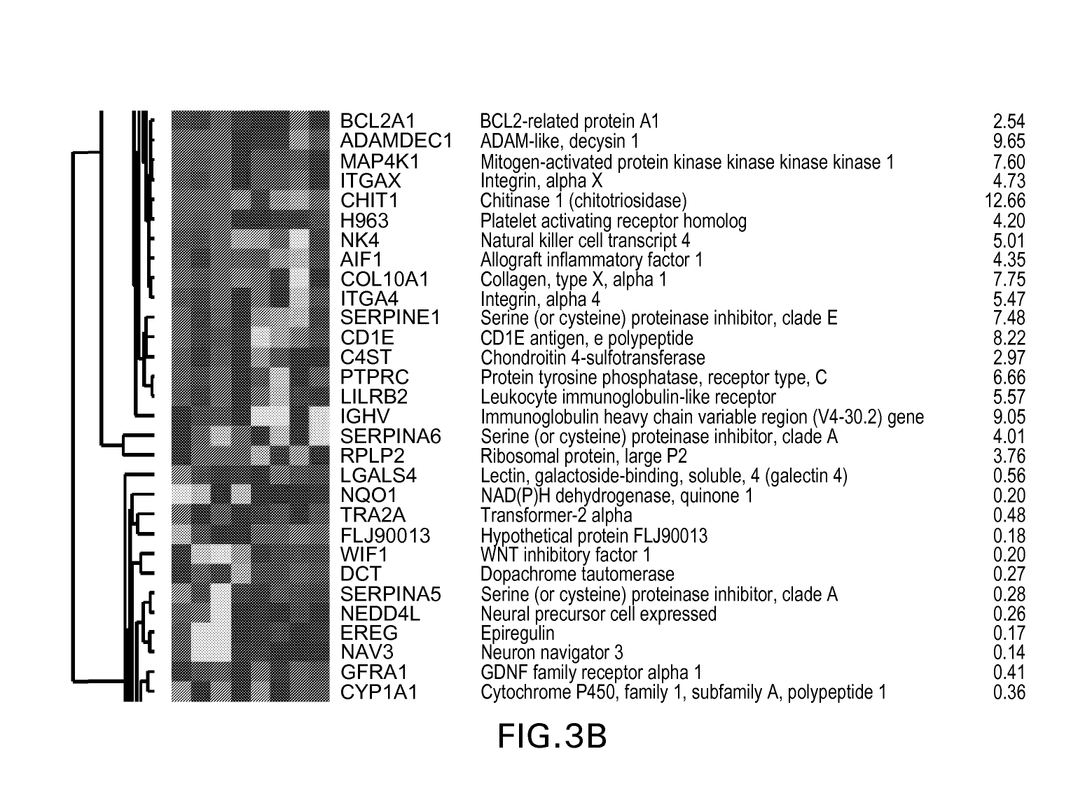

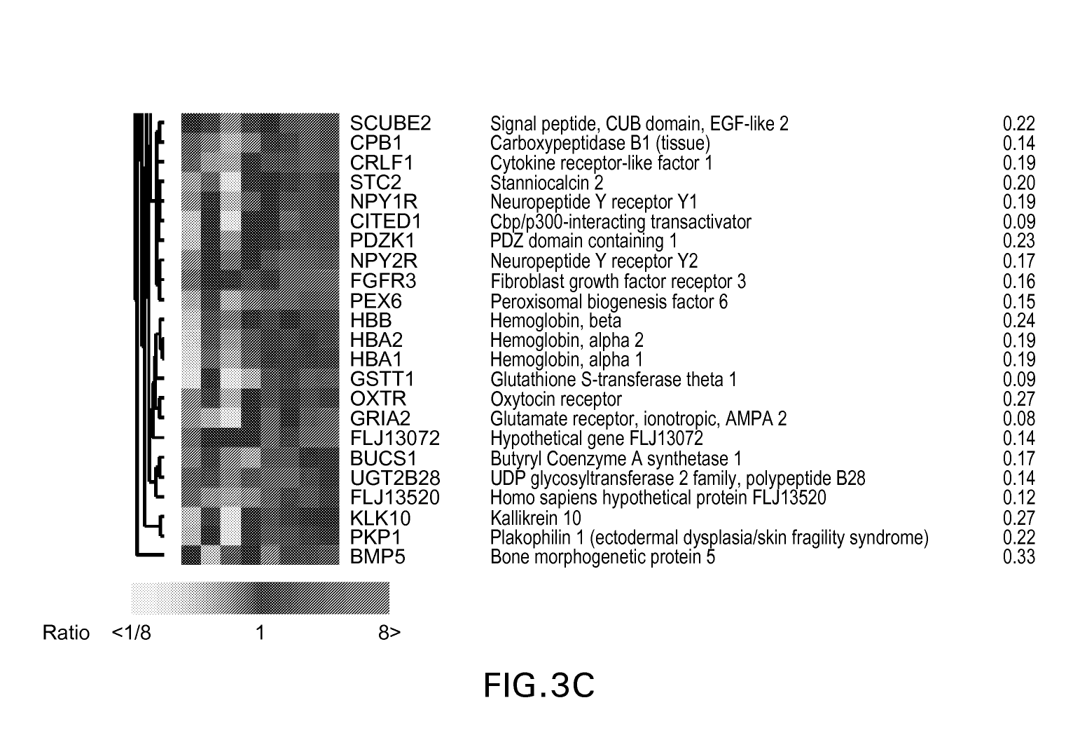

FIG. 2. Validation of gene expression by micro-arrays using quantitative real-time PCR. The expression of a set of 5 genes, CEA-CAM5, MMP-1, BCL2A, ER∃, and HEC was examined in a total of ten individual ADH, and six individual ADHC samples by RT quantitative real-time PCR using Assays-on-Demand™ reagents from ABI. Mean expression values are shown as histograms and standard deviation is shown as error bars. The real-time PCR data showed excellent concordance with the micro-array data establishing the authenticity of data by global gene expression analysis.

Hierarchical clustering of unique genes having top 35 and bottom 35 ADHC/ADH ratios and 5 interesting genes (BCL.2A1, BIRC1, TACC3, CEACAM5, and TYMS) by average linkage and centered correlation. Hierarchical clustering use 1-ρ as distance metric, where ρ is Pearson correlation coefficient is shown. Logarithmic signal values were mean centered for each gene. Each point in space represents one array. The cluster is color coded using red for up-regulation and green for down regulation and black for medium expression.

An aspect of the present invention provides a method of diagnosing breast cancer in a human patient. The level of gene expression is determined on a tissue sample obtained from a patient. Medical personnel, including biomedical researchers, are familiar with techniques to obtain samples of breast tissue. Tissue may be obtained by biopsy and lavage techniques. A determination is made of the level of expression of a least one gene upregulated in breast cancer. A determination may be made on a gene which is upregulated in pre-cancerous conditions. The gene upregulated in breast cancer or precancerous conditions may be termed a marker gene. Suitable, non-limiting marker genes include any or all of MMP-1, HEC, CEACAM5, BCL2A1, or any other gene shown to be upregulated in breast cancer or pre-cancerous conditions.

A preferred method of diagnosing breast cancer comprises detecting a differential level of expression of at least one marker gene relative to a housekeeping gene. A housekeeping gene may be defined as a gene whose level of expression is essentially invariant regardless of whether the tissue is in a normal, pre-cancerous, or cancerous state. A preferred, but not limiting, housekeeping gene is glyceraldehyde phosphate dehydrogenase (GAPDH).

A preferred method of diagnosing breast cancer in a human patient comprises contacting a gene array with a human breast tissue sample. According to an aspect, the gene array comprises at least one of the genes for MMP-1, HEC, CEACAM5, BCL2A1, or any other gene shown to be upregulated in breast cancer. Preferably, the gene array comprises a housekeeping gene. More preferably, the gene array comprises GAPDH. Preferably, the gene array comprises human genes. Persons of skill in the art know that suitable gene arrays, including human gene arrays may be obtained through commercial sources.

According to an alternative preferred aspect of the present invention, a method of diagnosing breast cancer in a human patient comprises contacting a nucleic acid extracted from a human breast tissue sample with a PCR reaction in the presence of at least one primer or probe specific for at least one of the genes for MMP-1, HEC, CEACAM5, BCL2A1, or any other gene shown to be upregulated in breast cancer. According to an aspect, the nucleic acid is an mRNA for at least one of the genes for MMP-1, HEC, CEACAM5, BCL2A1, or any other gene shown to be upregulated in breast cancer. Persons of skill in the art know that suitable probes and primers may be obtained from commercial sources.

A preferred embodiment provides a breast cancer-specific gene array chip. In a breast cancer-specific gene array chip, the genetic component of the gene array consists of genes that are upregulated in ADHC tissues and housekeeping genes. In a more preferred embodiment of a breast cancer-specific gene array chip, the genetic component of the gene array consists of the genes for MMP-1, HEC, CEACAM5, BCL2A1, and at least one housekeeping gene.

According to an aspect of the present invention, a method of diagnosing breast cancer in a human patient comprises contacting a nucleic acid reverse transcribed from a human breast tissue sample with a PCR reaction in the presence of at least one primer or probe specific for at least one of the genes for MMP-1, HEC, CEACAM5, BCL2A1, or any other gene shown to be upregulated in breast cancer. According to an aspect, the nucleic acid is an mRNA for at least one of the genes for MMP-1, HEC, CEACAM5, BCL2A1, or any other gene shown to be upregulated in breast cancer.

According to an aspect the present invention provides a method of monitoring the progress of a breast cancer clinical treatment. Aspects of the method comprise providing a patient with a clinical treatment, obtaining a tissue sample from said patient, and detecting the level of expression in a said tissue sample of a least one gene selected from the group consisting of MMP-1, HEC, CEACAM5, and BCL2A1, or any other gene shown to be upregulated in breast cancer. According to an aspect, a differential level of expression of at least one selected gene is detected in relation to the level of expression of a housekeeping gene.

According to an aspect the present invention provides a kit for diagnosing breast cancer comprising at least one component selected from the group consisting of a gene array chip, a breast cancer-specific gene array chip, a breast cancer-specific PCR primer, and a breast cancer-specific PCR probe. According to an aspect, the breast cancer-specific gene array chip comprises at least on gene selected from the group consisting of MMP-1, HEC, CEACAM5, and BCL2A1, or any other gene shown to be upregulated in breast cancer.

Micro-array data analysis and statistical calculations. All the micro-array analyses were performed at the Genome core facility, George Washington University Medical School, Washington, D.C. Partekpro 5.0, Partek, Inc. software was used for the data analyses. In order to identify differentially expressed genes between ADH and ADHC, logarithmic ratios (to the base 2) of ADHC/ADH were calculated by comparison analysis of MAS5 for all 16 combinations of ADHC and ADH pairs. The ratios associated with a “change” p-value<0.003 (increase) or >0.997 (Decrease) or a signal detection p-value <0.065 were included for further calculations. Mean of all ratios, one sample t-test p-value, number of Increase, and Decrease calls and sample size were calculated for each gene. Differentially expressed genes between ADHC and ADH samples were selected when mean ratio is 2-fold, p<0.001, at least 8 of the 16 ratio values were present and there was no tie between number of Increase and Decrease calls. At the above statistical significance level, we observed a total of 540 differentially altered genes in ADHC. At a mean ratio of 2-fold up, 371 were up-regulated and 169 down-regulated in ADHC. Over-representation analysis of gene ontologies was performed on the genes that had ontology annotations using Expression Analysis Systematic Explorer (EASE) software. (Hosack, et al., 2003)

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

### REFERENCES

1. Jemal, A. et al., 56 CA Cancer J. Clin. 10-30 (2005).
2. Allred, D. C., Mohsin, S. K. & Fuqua, S. A. W., 8 Endocr. Relat. Cancer 47-61 (2001).
3. Krishnamurthy, S. & Sneige, N., 9 Adv. Anat. Pathol. 185-197 (2002).
4. Lacroix, M., Zammatteo, N., Remacle, J. & Leclercq, G., 17 Int. J. Biol. Markers 5-23 (2002).
5. Ma, X.-J. et al., 100 Proc. Natl. Acad. Sci. USA 5974-5979 (2003).
6. Migita, T. et al., 84 Int. J. Cancer 74-79 (1999).
7. Balduyck, M. et al., 18 Clin. Exp. Metastasis 171-178 (2000).
8. Hojilla, C. V., Mohammed, F. F. & Khokha, R., 89 Br. J. Cancer 1817-1821 (2003).
9. Hosack, D. A., Dennis, G., Sherman, B. T., Lane H. C. and Lempicki, R. A. (2003) “Identifying biological themes within lists of genes with EASE,” Genome Biol. 4, R70.

### INCORPORATION BY REFERENCE

Throughout this application, various references including publications, patents, and pre-grant patent application publications are referred to. Disclosures of these publications in their entireties are hereby incorporated by reference into this application to more fully describe the state of the art to which this invention pertains. It is specifically not admitted that any such reference constitutes prior art against the present application or against any claims thereof. All publications, patents, and pre-grant patent application publications cited in this specification are herein incorporated by reference, and for any and all purposes, as if each individual publication or patent application were specifically and individually indicated to be incorporated by reference. In the case of inconsistencies the present disclosure will prevail. The various published work of the inventor is hereby incorporated by reference. Works of the inventor specifically incorporated by reference include, but are not limited to: Poola, I (2003) Molecular Assay To Generate Expression Profile Of Eight Estrogen Receptor Alpha Isoform mRNA Copy Numbers In Picogram Amounts Of Total RNA From Breast Cancer Tissues. Anal. Biochem. 314, 217-226; and Poola I, et al., Identification of MMP-1 as a Putative Breast Cancer Predictive Marker By Global Gene Expression Analysis, 11 Nature Medicine, 481, (1 May 2005), the entire contents of which is expressly incorporated by reference for all purposes. This incorporation includes any and all supplementary materials published online.

### ELECTRONIC SUBMISSION OF COMPLEX WORK UNITS

For convenience of the Examiner, the large Figures and Tables are being submitted as electronic files. The electronic submission is in addition to the paper copy. The paper and electronic forms comprise the same subject matter.

Having thus described our invention, what we claim as new, and desire to secure by Letters Patent is:

1. A method of diagnosing breast cancer in a human patient, comprising detecting the level of expression in a tissue sample of a least one gene selected from the group consisting of MMP-1, HEC, CEACAM5, and BCL2A1.

2. The method of diagnosing breast cancer in a human patient, according to claim 1, wherein said detecting comprises detecting a differential level of expression of said at least one selected gene relative to a housekeeping gene.

3. The method of diagnosing breast cancer in a human patient, according to claim 1, wherein said detecting comprises contacting a human gene array with said tissue sample.

4. The method of diagnosing breast cancer in a human patient, according to claim 1, wherein said detecting comprises detecting at least one mRNA by PCR.

5. A method of monitoring the progress of a breast cancer clinical treatment comprising:
providing a patient with a clinical treatment;
obtaining a tissue sample from said patient; and
detecting the level of expression in a said tissue sample of a least one gene selected from the group consisting of MMP-1, HEC, CEACAM5, and BCL2A1.

6. The method of monitoring the progress of a breast cancer clinical treatment, according to claim 5, wherein said detecting comprises detecting a differential level of expression of said at least one selected gene relative to a housekeeping gene.

7. The method of monitoring the progress of a breast cancer clinical treatment, according to claim 5, wherein said detecting comprises contacting a human gene array with said tissue sample.

8. The method of monitoring the progress of a breast cancer clinical treatment, according to claim 5, wherein said detecting comprises detecting at least one mRNA by PCR.

9. A kit for diagnosing breast cancer comprising at least one component selected from the group consisting of a gene array chip, a breast cancer-specific gene array chip, a breast cancer-specific PCR primer, and a breast cancer-specific PCR probe.

10. The kit for diagnosing breast cancer, according to claim 9, wherein said breast cancer-specific gene array chip comprises at least one gene selected from the group consisting of MMP-1, HEC, CEACAM5, and BCL2A1.

11. A breast cancer-specific gene array chip comprising at least one housekeeping gene and at least one gene selected from the group consisting of MMP-1, HEC, CEACAM5, and BCL2A1.
